(19) United States
(12) Patent Application Publication
(10) Pub. No.: US 2019/0015484 A1
(43) Pub. Date: Jan. 17, 2019

(54) NICOTINE-DEGRADING ENZYMES FOR TREATING NICOTINE ADDICTION AND NICOTINE POISONING

(71) Applicants: The Scripps Research Institute, La Jolla, CA (US); Antidote Therapeutics, Inc., Gaithersburg, MD (US)

(72) Inventors: Kim D. JANDA, La Jolla, CA (US); Matt KALNIK, Gaithersburg, MD (US); Thomas THISTED, Gaithersburg, MD (US)

(73) Assignees: The Scripps Research Institute, La Jolla, CA (US); Antidote Therapeutics, Inc., Gaithersburg, MD (US); Antidote Therapeutics, Inc., Gaithersburg, MD (US)

(21) Appl. No.: 15/749,630

(22) PCT Filed: Aug. 2, 2016
(86) PCT No.: PCT/US2016/045109

§ 371 Date: Feb. 1, 2018

Related U.S. Application Data
(60) Provisional application No. 62/200,968, filed on Aug. 4, 2015.

(51) Int. Cl. A61K 38/44; A61K 47/60; C12N 9/02; A61P 25/34; A61K 38/16; A61K 47/68

(52) U.S. Cl. CPC A61K 38/44; A61K 47/60; C12N 9/0093; C12Y 101/00; A61K 38/164; A61K 47/68; A61P 25/34

(57) ABSTRACT

Described herein are methods and compositions for treating nicotine addiction, promoting smoking cessation, reducing the risk of relapse of nicotine consumption, and/or treating nicotine poisoning in a subject in need thereof, using a nicotine-degrading enzyme or an expression vector capable of expressing a nicotine-degrading enzyme in vivo.

### RELATED APPLICATIONS

This application claims priority benefits under 35 U.S.C. § 119(e) to U.S. Provisional Application No. 62/200,968, filed Aug. 4, 2015, the entire contents of which are incorporated herein by reference in their entirety.

### FIELD

Described herein are methods and compositions for treating nicotine addiction, promoting smoking cessation, reducing the risk of relapse of nicotine consumption, and/or treating nicotine poisoning in a subject in need thereof, using a nicotine-degrading enzyme or an expression vector capable of expressing a nicotine-degrading enzyme in vivo.

### BACKGROUND

Tobacco use continues to be one of the leading causes of preventable death, indeed; approximately 6 million mortalities are attributed to nicotine use.¹ Most smokers are aware of the health consequences of smoking, and while they want to quit, abstinence is usually difficult to maintain.² The current pharmacological aids used in smoking cessation can have significant clinical effects. Representative examples include nicotine replacement therapies,³ the antidepressant drug bupropion,⁴ and the recently introduced varenicline, which have all shown success in increasing abstinence rates compared to placebo.⁵ Still, even with these pharmacological aids, the majority of the smokers and their long-term success rates remain low as only 15-30% of smokers remain abstinent for at least 1 year after treatment, therefore alternative therapies are needed.⁶

The inventor, as well as other workers, have pursued a pharmacokinetic (antibody-based) as opposed to a pharmacodynamic (drug-based) strategy to aid in smoking abstinence.⁷ Nicotine vaccines have been proposed to provide long-lasting protection, and to date multiple nicotine vaccines have advanced into clinical trials.⁸ However, these vaccines typically have limited efficacy or they have failed to achieve their primary end-point of increased smoking cessation rates compared to placebo.⁸⁻⁹ Thus, while past studies on antibody sequestration of nicotine delivered a proof-of-concept that a pharmacokinetic strategy can enhance smoking cessation rates,¹⁰ it is also apparent that higher concentrations of antibody are needed to make an effective vaccine.^{10b}

### SUMMARY

In accordance with some aspects, provided herein are methods of treating nicotine addiction, promoting smoking cessation, reducing the risk of relapse of nicotine consumption, or treating nicotine poisoning in a subject in need thereof, comprising administering to the subject a therapeutically effective amount of a nicotine-degrading enzyme.

In some embodiments, the nicotine-degrading enzyme degrades nicotine into a compound selected from the group consisting of N-methylmyosmine and 4-(methylamino)-1 (pyridine-3-yl)butan-1-one.

In specific embodiments, the nicotine-degrading enzyme is obtained from Pseudomonas putida. In further specific embodiments, the nicotine-degrading enzyme is NicA2 or a NicA2 variant that exhibits nicotine-degrading activity in vivo. In further specific embodiments, the amino acid sequence of NicA2 corresponds to SEQ ID NO:1. In further specific embodiments, the amino acid sequence of the NicA2 variant is at least 95% identical to SEQ ID NO:1. In specific embodiments, the amino acid sequence of the NicA2 variant is modified as compared to SEQ ID NO:1 to reduce immunogenicity in the subject, to enhance catalytic efficiency of the enzyme, and/or to enhance stability of the enzyme.

In some embodiments, the nicotine-degrading enzyme is conjugated or fused to a moiety that increases the circulating half-life of the enzyme in vivo. In specific embodiments, the moiety is selected from the group consisting of polyethylene glycol moieties, albumin moieties, and albumin-binding moieties. In further specific embodiments, the moiety may include an antibody Fc domain and/or a peptide moiety that mimics the half-life extending properties of polyethylene glycol

In some embodiments, the method comprises administering the enzyme by a route of administration selected from the group consisting of intranasally, orally, subcutaneously, intravenously, intraperitoneally, and intramuscularly.

In some embodiments, the method comprises administering an amount of nicotine-degrading enzyme of from 0.01 mg/kg to 100 mg/kg. In some embodiments, the method comprises administering an amount of nicotine-degrading enzyme effective to achieve a serum concentration of nicotine-degrading enzyme of from about 0.1 μM to about 50 μM. In some embodiments, the method comprises administering an amount of nicotine-degrading enzyme effective to achieve a serum concentration of from about 0.5 μM to about 10 μM, including about 4 μM, of the nicotine-degrading enzyme.

In some embodiments, the method is effective to reduce serum levels of nicotine in the subject. In some embodiments, the method is effective to reduce brain levels of nicotine in the subject.

In some embodiments, the nicotine-degrading enzyme is administered once daily, once every two days, once every three days, twice weekly, once weekly, once every two weeks, once every three weeks, once every month, once every two months, once every three months, once every six months, or less frequently.

In some embodiments, the method is effective to treat nicotine addiction, treat a nicotine-addiction related disorder, reduce the risk of relapse of nicotine consumption, promote smoking cessation, extend a duration of smoking abstinence in a subject who has quit smoking, increase a likelihood of long-term abstinence from smoking, and/or rescue a subject from relapse of nicotine consumption. In some embodiments, the method is effective to treat nicotine poisoning in the subject.

In accordance with other aspects, provided herein are methods of degrading nicotine in vivo in a subject in need thereof, comprising administering to the subject a therapeutically effective amount of a nicotine-degrading enzyme.

In accordance with other aspects, provided herein are methods of degrading nicotine in vivo in a subject in need thereof, comprising administering to the subject a therapeutically effective amount of an expression vector capable of expressing a nicotine-degrading enzyme in vivo.

In accordance with other aspects, provided herein are pharmaceutical compositions comprising a therapeutically effective amount of a nicotine-degrading enzyme as described herein in a pharmaceutically acceptable carrier. In some embodiments, the composition is formulated for administration by a route selected from the group consisting of intranasally, orally, subcutaneously, intravenously, intraperitoneally, and intramuscularly.

Also provided are pharmaceutical compositions as described herein for use in treating nicotine addiction, promoting smoking cessation, reducing the risk of relapse of nicotine consumption, and/or treating nicotine poisoning in a subject in need thereof.

Also provided are uses of a pharmaceutical composition as described herein in the preparation of a medicament for treating nicotine addiction, promoting smoking cessation, reducing the risk of relapse of nicotine consumption, and/or treating nicotine poisoning in a subject in need thereof.

### DESCRIPTION OF THE FIGURES

FIG. 1 shows NicA2 protein electrophoretically separated on SDS-polyacrylamide gel, illustrating the purity and the molecular weight of the NicA2 preparation.

FIG. 2 shows the products of NicA2 as detected by LC-MS. The product with m/z=179 had retention time 2.7 min and the double peak with m/z=161 had retention times of 2.1 and 2.6 min.

FIG. 3 illustrates standard curve generation based upon m/z peak area of 161 (1 and 2) or 179 (4) versus a decrease in nicotine concentration.

FIGS. 4A-4B illustrate the kinetics of NicA2 degradation of nicotine. FIG. 4A shows a Michaelis-Menten curve of NicA2 based on the amounts of m/z 179 or 161 formed. FIG. 4B shows a Michaelis-Menten curve of NicA2 activity at 37° C. compared to a Michaelis-Menten curve of NicA2 activity at room temperature. The following Michaelis-Menten parameters were obtained for NicA2 activity at 37° C.: Kₘ=91.9±10.4 nM, k_{cat}=(1.32±0.04)×10⁻² s⁻¹ and k_{cat}/Kₘ=1.44×10⁵ s⁻¹·M⁻¹ (see Table 1 for comparison to room temperature).

FIGS. 5A-5D illustrate the stability of the NicA2 enzyme. FIG. 5A graphically illustrates the thermal stability of the NicA2 enzyme as a function of temperature. FIG. 5B illustrates the stability of NicA2 at 37° C. in HEPES buffer (pH=7.4) as a function of time. Note the v₀ remained virtually constant over the duration of the study. FIG. 5C illustrates the stability of NicA2 in mouse serum at 37° C. over time. FIG. 5D graphically illustrates the ability of NicA2 to degrade nicotine in mouse serum. Concentrations of 125, 250, 500 nM nicotine with and without enzyme (20 nM NicA2) were incubated in serum for 30 min. Residual nicotine remaining after this time period was measured.

FIG. 6 graphically illustrates a simulation of blood nicotine concentrations over time after one cigarette and administration of 20 nM NicA2, and shows that the NicA2 enzyme reduces the nicotine half-life to 9-15 min.

FIG. 7 shows an ultraviolet (UV)-visible absorbance spectrum of NicA2. The minor peak at 370 nm and major peak at 450 nm match what is typically seen with a flavin protein.

FIG. 8 graphically illustrates that the addition 40 μM FMN or FAD did not affect the activity of NicA2.

FIGS. 9A-9B graphically illustrate reduction of blood and brain levels of nicotine to below the limit of quantitation in vivo in rats at 5 minutes after 0.03 mg/kg i.v. nicotine dose (n=5).

FIG. 10 graphically shows that an about 10 mg/kg dose of NicA2 is effective to reduce brain nicotine levels by greater than 95%.

### DETAILED DESCRIPTION

Described herein are compositions and methods for degrading nicotine, treating nicotine addiction, promoting smoking cessation, reducing the risk of relapse of nicotine consumption, and/or treating nicotine poisoning in a subject in need thereof comprising administering a therapeutically effective amount of a nicotine-degrading enzyme.

### Definitions

Technical and scientific terms used herein have the meanings commonly understood by one of ordinary skill in the art of to which the present disclosure pertains, unless otherwise defined. Reference is made herein to various methodologies known to those of ordinary skill in the art. Suitable materials and/or methods known to those of ordinary skill in the art can be utilized in carrying out the present disclosure. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, the term “about” means that the number or range is not limited to the exact number or range set forth, but encompass values around the recited number or range as will be understood by persons of ordinary skill in the art depending on the context in which the number or range is used. Unless otherwise apparent from the context or convention in the art, “about” means up to plus or minus 10% of the particular term.

As used herein, “subject” denotes any mammal, including humans. For example, a subject may be suffering from or at risk of developing a condition that can be diagnosed, treated or prevented with a nicotine-degrading enzyme.

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject.

The terms “treat”, “treating” or “treatment”, as used herein, include alleviating, abating or ameliorating a disease or condition or one or more symptoms thereof, whether or not the disease or condition is considered to be “cured” or “healed” and whether or not all symptoms are resolved. The terms also include reducing or preventing progression of a disease or condition or one or more symptoms thereof, impeding or preventing an underlying mechanism of a disease or condition or one or more symptoms thereof, and achieving any therapeutic and/or prophylactic benefit.

As used herein, the phrase “therapeutically effective amount” refers to a dose that provides the specific pharmacological effect for which the drug is administered in a subject in need of such treatment. It is emphasized that a therapeutically effective amount will not always be effective in treating the conditions described herein, even though such dose is deemed to be a therapeutically effective amount by those of skill in the art. For convenience only, exemplary doses and therapeutically effective amounts are provided below with reference to adult human subjects. Those skilled in the art can adjust such amounts in accordance with standard practices as needed to treat a specific subject and/or condition/disease.

### Effects of Smoking

In humans, nicotine is absorbed rapidly from cigarette smoke, from which it enters the arterial circulation through the oral mucosa and lungs and is rapidly distributed to body tissues.¹² It takes approximately 20 seconds for nicotine to pass through the brain.¹³ While the elimination half-life that is relevant to the accumulation of nicotine during the use of tobacco averages 2-3 hours.¹² Thus, nicotine levels accrue over 6-8 hours during regular smoking and there is a long terminal half-life, 20 hours or more, presumably reflecting the slow release of nicotine from tissue.¹² Moreover, smoking represents a multiple dosing situation with considerable accumulation while smoking and persistent levels for 24 hours of each day.

The metabolism of nicotine in mammals involves at least two degradation pathways, as shown below.

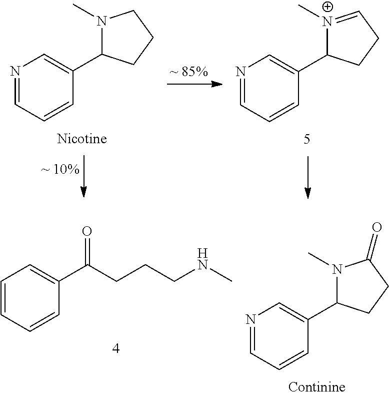

As illustrated, there are at least two unique nicotine degradation pathways in mammals, one producing cotinine and the second 4-(methylamino)-1-(pyridine-3-yl)butan-1-one (4). Cotinine and its metabolites account for 70-80% of nicotine metabolites in humans while the aminoketone 4 is a minor component in humans.¹² Cotinine has been shown to be pharmacologically active, and some of nicotine's effects in the nervous system may be mediated by cotinine and/or complex interactions with nicotine itself (Grizzell & Echeverria, Neurochem Res 27 (2014); Crooks & Dwoskin, Biochem Pharm 1(54): 743-53 (1997)).

As explained below, it is this latter pathway to compound 4 that raised the tantalizing possibility of an alternative to the nicotine immunotherapy¹⁴. In particular, it was surprisingly discovered that a nicotine-degrading enzyme can be used to degrade nicotine in vivo and thereby treat nicotine addiction, promote smoking cessation, reduce the risk of relapse of nicotine consumption, and/or treat nicotine poisoning in a subject in need thereof.

### Methods for Degrading Nicotine In Vivo

As noted above, described herein are methods and compositions for degrading nicotine in vivo. The methods comprise administering to a subject in need thereof a nicotine-degrading enzyme or an expression vector capable of expressing a nicotine-degrading enzyme in vivo. The compositions comprise the nicotine-degrading enzyme and/or expression vector, optionally together with a pharmaceutically acceptable carrier. The methods and compositions are useful for treating nicotine addiction, promoting smoking cessation, reducing the risk of relapse of nicotine consumption, and/or treating nicotine poisoning in a subject in need thereof.

The subject can be a mammal, including a human or other animal, such as a human or other animal in need of nicotine detoxification, in need of reduction of nicotine's psychoactive effects, or in need of treatment for the addictive effects of nicotine, or in need of treatment for any of the other conditions discussed herein.

In specific embodiments, the method treats nicotine addiction in a subject in need thereof. In specific embodiments, the method promotes smoking cessation in a subject in need thereof. In specific embodiments, the method reduces the relapse of nicotine consumption in a subject in need thereof. In specific embodiments, the method treats nicotine poisoning in a subject in need thereof. In specific embodiments, the method is effective to treat nicotine addiction. In specific embodiments, the method is effective to treat a nicotine-addiction related disorder. In specific embodiments, the method is effective to reduce the risk of relapse of nicotine consumption. In specific embodiments, the method is effective to promote smoking cessation. In specific embodiments, the method is effective to extend a duration of smoking abstinence in a subject who has quit smoking. In specific embodiments, the method is effective to increase a likelihood of long-term abstinence from smoking. In specific embodiments, the method is effective to rescue a subject from relapse of nicotine consumption. In specific embodiments, the method is effective to treat nicotine poisoning.

A therapeutically effective amount of the nicotine-degrading enzyme or expression vector therefor may depend on the subject being treated, the condition being treated, the desired effect, and the intended duration of the therapeutic effect. A therapeutically effective amount of the nicotine-degrading enzyme or expression vector therefor may be from about 0.01 mg/kg to about 100 mg/kg, including any amount in between. Accordingly, in specific embodiments, the method comprises administering from about 0.01 mg/kg to about 100 mg/kg, or any amount in between, or greater, of the nicotine-degrading enzyme or expression vector therefor. For example, the method may comprise administering from about 0.01 mg/kg to about 500 to 750 mg/kg, about 0.01 mg/kg to about 300 to 500 mg/kg, about 0.1 mg/kg to about 100 to 300 mg/kg or about 1 mg/kg to about 50 to 100 mg/kg of body weight, of the nicotine-degrading enzyme or expression vector therefor although other dosages may provide beneficial results. The amount administered may be adjusted depending on various factors including, but not limited to, the specific enzyme, nucleic acid, vector or combination thereof being administered (including whether it is modified to enhance efficacy and/or prolong half-life); the disease or condition being treated; the weight of the subject; the physical condition of the subject (including the degree of smoking addiction, level of circulating nicotine, etc.), the health of the subject, and the age of the subject. Such factors can be determined by employing animal models, clinical trials, or other test systems available in the art.

In specific embodiments, the amount of enzyme administered may be from about 0.5 mg/kg to about 100 mg/kg, from about 10 mg/kg to about 100 mg/kg, from about 20 mg/kg to about 100 mg/kg, from about 30 mg/kg to 100 mg/kg, from 40 mg/kg to 100 mg/kg, from 50 mg/kg to 100 mg/kg, from 60 mg/kg to 100 mg/kg, from 70 mg/kg to 100 mg/kg, or from 80 mg/kg to 100 mg/kg of the nicotine-degrading enzyme per body weight of the subject. In other specific embodiments, the method may comprise administering from 10 mg/kg to 90 mg/kg, from 20 mg/kg to 80 mg/kg, from 30 mg/kg to 70 mg/kg, or from 40 mg/kg to 60 mg/kg of the nicotine-degrading enzyme per body weight of the subject. In further specific embodiments, the method may comprise administering 0.01 mg/kg, 0.1 mg/kg, 0.5 mg/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 55.0 mg/kg, 60.0 mg/kg, 65.0 mg/kg, 70.0 mg/kg, 75.0 mg/kg, 80.0 mg/kg, 85.0 mg/kg, 90.0 mg/kg, 95.0 mg/kg, or 100.0 mg/kg of the nicotine-degrading enzyme per body weight of the subject. These amounts are based on the weight of the nicotine-degrading enzyme; thus, if the enzyme is conjugated or fused to another moiety as discussed in more detail below, higher amounts of active agent may be administered,

Daily doses of the nicotine-degrading enzyme can vary as well, in accordance with these ranges and other factors discussed herein. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day. Similar doses may be used for weekly, monthly, or less frequent dosing, depending on the half-life of the nicotine-degrading enzyme construct administered.

In some embodiments, the therapeutically effective amount of the nicotine-degrading enzyme or expression vector therefor administered achieves a serum concentration of the nicotine-degrading enzyme of from about 20 nM to about 400 nM in the subject. In further specific embodiments, the therapeutically effective amount of the nicotine-degrading enzyme or expression vector therefor may achieve a serum concentration of at least 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, or 400 nM of the nicotine-degrading enzyme in the subject. In further specific embodiments, the therapeutically effective amount of the nicotine-degrading enzyme or expression vector therefor achieves a serum concentration of at least 20 nM of the nicotine-degrading enzyme.

In other embodiments, the therapeutically effective amount of the nicotine-degrading enzyme or expression vector therefor administered achieves a serum concentration of the nicotine-degrading enzyme of from about 0.1 μM to about 100 μM, or from about 0.1 μM to about 50 μM, or from about 0.2 μM to about 50 μM, or from about 0.4 μM to about 40 μM, or from about 0.5 μM to about 10 μM in the subject. For example, the therapeutically effective amount of the nicotine-degrading enzyme or expression vector therefor administered may achieve a serum concentration of at least 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 2.0 μM, 2.5 μM, 3.0 μM, 3.5 μM, 4.0 μM, 4.5 μM, 5.0 μM, 6.0 μM, 7.0 μM, 8.0 μM, 9.0 μM, 10.0 μM, 12.0 μM, 14.0 μM, 16.0 μM, 18.0 μM, 20.0 μM, 22.0 μM, 25.0 μM, 28.0 μM, 30.0 μM, 32.0 μM, 35.0 μM, 38.0 μM, 40.0 μM, 42.0 μM, 45.0 μM, 48.0 μM, or 50.0 μM, of the nicotine-degrading enzyme in the subject. In further specific embodiments, the therapeutically effective amount of the nicotine-degrading enzyme or expression vector therefor achieves a serum concentration of at least 0.4 μM of the nicotine-degrading enzyme.

Plasma levels of nicotine in smokers are typically from about 25 to about 300 nM, or from about 5 to about 60 ng/ml. Arterial levels of nicotine following one puff from a cigarette are typically about 7 ng/ml, and after smoking a cigarette are typically in the 10-20 ng/ml range. Thus, in some embodiments, the therapeutically effective amount of nicotine-degrading enzyme is effective to degrade about 1 to about 300 nM, or from about 25 to about 300 nM, or from about 5 to about 60 ng/ml, nicotine, or to reduce serum nicotine levels to below 200 nM, below 100 nM, below 60 nM, below 50 nM, below 40 nM, below 20 nM, below 10 nM, below 5 nM, below 1 nM, below 0.5 nM, below 0.1 nM, below 0.05 nM, or below 0.001 nM. Additionally or alternatively, in some embodiments, the amount of nicotine-degrading enzyme or expression vector therefor administered is effective to reduce the effect of nicotine at neuronal nicotinic acetylcholine receptors (nAChR) in the subject, such as to reduce plasma and/or brain levels of nicotine to levels below the nicotine Kᵢ for nAChr (such as below 1-12 nM), and/or below the nicotine EC50 for activation of nAChr (such as below 60 nM) and/or below the nicotine EC50 for desensitization of nAChr (such as below 2.8 nM). Additionally, or alternatively, in some embodiments, the amount of nicotine-degrading enzyme or expression vector therefor administered reduces the effect of nicotine at nAChRs by at least 20%, at least 25%, at least 30%, at least 50%, at least 75%, at least 90%, or at least 95%.

As noted above, in specific embodiments, the therapeutically effective amount is effective to treat nicotine addiction, promote smoking cessation, reduce the relapse of nicotine consumption, and/or treat nicotine poisoning in a subject in need thereof. In other specific embodiments, the therapeutically effective amount is effective to treat a nicotine addiction, treat a nicotine addiction related disorder, reduce the risk of relapse of nicotine consumption, promote smoking cessation, extend a duration of smoking abstinence in a subject who has quit smoking, increase a likelihood of long term abstinence from smoking, and/or rescue a subject from relapse of nicotine consumption.

The dosing frequency may be selected and adjusted depending on various factors including, but not limited to, the specific enzyme, nucleic acid, vector or combination thereof being administered (including whether it is modified to enhance efficacy and/or prolong half-life); the disease or condition being treated; the weight of the subject; the physical condition of the subject (including the degree of smoking addiction, level of circulating nicotine, etc.), the health of the subject, and the age of the subject. In specific embodiments, a therapeutically effective amount of the nicotine-degrading enzyme is administered once daily, once every two days, once every three days, twice weekly, thrice weekly, once weekly, once every two weeks, once every three weeks, once every month, or once every two months, once every three months, once every six months, or less frequently. In other specific embodiments, a therapeutically effective amount of the nicotine-degrading enzyme is administered several times a day.

In specific embodiments, administration of the nicotine-degrading enzyme or expression vector therefor is in a single dose, in multiple doses, in a continuous or intermittent manner, depending, for example, upon the recipient's physiological condition, whether the purpose of the administration is therapeutic or prophylactic, and other factors known to skilled practitioners. The administration of the nicotine-degrading enzymes, expression vectors, and compositions may be essentially continuous over a preselected period of time or may be in a series of spaced doses. Both local and systemic administration is contemplated.

In some embodiments, the method is effective to reduce nicotine levels in the subject. In specific embodiments, the method is effective to reduce serum levels of nicotine in the subject. In specific embodiments, the method is effective to reduce serum levels of nicotine in the subject by at least 10%, 15%, 20%, 25%, 30%, 35%, 40%, 45%, 50%, 55%, 60%, 65%, 70%, 75%, 80%, 85%, 90%, 95%, 96%, 97%, 98%, or 99%, including by 10%, 20%, 30%, 40%, 50%, 60%, 70%, 80%, 90%, 95%, 96%, 97%, 98%, 99%, or more. In other specific embodiments, the method is additionally or alternatively effective to reduce brain levels of nicotine in the subject. In specific embodiments, the method is additionally or alternatively effective to reduce brain levels of nicotine in the subject by at least 10%, 15%, 20%, 25%, 30%, 35%, 40%, 45%, 50%, 55%, 60%, 65%, 70%, 75%, 80%, 85%, 90%, 95%, 96%, 97%, 98%, or 99%, including by 10%, 20%, 30%, 40%, 50%, 60%, 70%, 80%, 90%, 95%, 96%, 97%, 98%, 99%, or more. In some embodiments, a higher dose is needed to achieve greater than 95% reduction of brain levels of nicotine as compared to that effective to achieve greater than 95% reduction of serum levels of nicotine, such as 2×, 4×, 8×, 10×, 20×, 30×, 40×, 50×, or 100× of a dose effective to achieve greater than 95% reduction of serum levels.

The nicotine-degrading enzyme or expression vector therefor may be administered by any a route of administration. In specific embodiments, the nicotine-degrading enzyme is administered by a route of administration selected from the group consisting of intranasally, orally, subcutaneously, intravenously, intraperitoneally, and intramuscularly. In specific embodiments, the nicotine-degrading enzyme and/or expression vector is formulation in a pharmaceutical composition suitable for the intended route of administration, as discussed in more detail below.

Therefore, in accordance with one aspect, provided herein is a method that involves administering at least one nicotine-degrading enzyme, or expression vector therefor, or a composition thereof, to a subject. Such a method can thereby degrade nicotine in the subject. For example, such a method can degrade more nicotine in a subject than a method where at least one nicotine-degrading enzyme, or a composition thereof, is not administered to a subject. In some embodiments, the nicotine-degrading enzyme is NicA2, which is described in more detail below.

In another aspect, provided herein is a method for reducing the incidence of nicotine addiction in a subject, where the method involves administering to the subject at least one nicotine-degrading enzyme or expression vector therefor or a composition thereof, to a subject, to thereby reduce the incidence of nicotine addiction in a subject. For example, the incidence of nicotine addiction is reduced in a subject by such a method, compared to a method where at least one nicotine-degrading enzyme, or a composition thereof, is not administered to a subject. The at least one nicotine-degrading enzyme or composition thereof can be administered prior to intake of nicotine, or during intake of nicotine. In some embodiments, the nicotine-degrading enzyme is NicA2.

In another aspect, provided herein is a method for reducing the toxicity of nicotine in a subject, where the method involves administering to the subject at least one nicotine-degrading enzyme or expression vector therefor or a composition thereof, to a subject, to thereby reduce the toxicity of nicotine in a subject. Such a method reduces the incidence of nicotine addiction in a subject, compared to a method where at least one nicotine-degrading enzyme, or a composition thereof, is not administered to a subject. In some embodiments, the nicotine-degrading enzyme is NicA2.

Thus, described herein are compositions and methods that enhance nicotine degradation. Such compositions and methods have utility for ameliorating the negative effects of nicotine absorption that occurs in people who smoke or chew tobacco. By decreasing the amount of nicotine in circulation throughout the body, the toxicity and psychoactive effects of nicotine are reduced. Hence, the methods and compositions of the invention can lower the amount of nicotine that reaches or is maintained in the brain, liver, and vascular system, thereby reducing the destructive physiological effects of nicotine.

### Compositions Comprising Nicotine-Degrading Enzymes or Vectors

As noted above, the nicotine-degrading enzyme and/or expression vector therefor may be formulated in a pharmaceutical composition suitable for the intended route of administration. Such compositions typically comprise a therapeutically effective amount of a nicotine-degrading enzyme or expression vector in a pharmaceutically acceptable carrier.

The carrier may be any pharmaceutically acceptable carrier. By “pharmaceutically acceptable carrier” it is meant a carrier, diluent, or excipient, that is compatible with the other ingredients of the formulation, and not deleterious to the subject.

To prepare a composition suitable for use in the methods described herein, enzymes, nucleic acids, vectors, and/or combinations thereof, and other agents (such as a pharmaceutically acceptable carrier) are synthesized or otherwise obtained, purified as necessary or desired and stabilized. For example, some of the enzymes, nucleic acids, vectors, combinations thereof, and other agents can be lyophilized. These agents can then be adjusted to the appropriate concentration, and optionally combined with other agents.

The absolute weight of a given enzyme, nucleic acid, vector, and/or other agent included in a unit dose can vary widely. For example, from about 0.01 to about 2 g, or from about 0.1 to about 500 mg, of at least one enzyme, nucleic acid, or vector as described herein, or a plurality or combination of enzymes, nucleic acids, vectors, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Thus, one or more suitable unit dosage forms comprising the enzymes, nucleic acids, vectors, and/or other agents can be administered by a variety of routes including parenteral (including subcutaneous, intravenous, intramuscular and intraperitoneal), oral, rectal, dermal, transdermal, intrathoracic, intrapulmonary and intranasal (respiratory) routes. The enzymes, nucleic acids, vectors, added agents, or combinations thereof may also be formulated for sustained release (for example, using microencapsulation, see WO 94/07529, and U.S. Pat. No. 4,962,091) or in depot formulations.

The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

Compositions suitable for use in the methods described herein may be prepared any suitable form, including aqueous solutions, suspensions, tablets, hard or soft gelatin capsules, and liposomes and other slow-release formulations, such as shaped polymeric gels. Administration of enzymes, nucleic acids, and/or expression vectors often involves parenteral or local administration in an aqueous solution or sustained release vehicle.

Enzymes, nucleic acids, vectors, and/or additional agents administered in an oral dosage form, may be formulated such that the enzyme, nucleic acid, vector, or additional agent is released into the intestine after passing through the stomach. Such formulations are described in U.S. Pat. No. 6,306,434 and in the references contained therein.

In specific embodiments for oral administration, the nicotine-degrading enzyme may be formulated to protect it from degradation, including proteolysis. Methods of formulating proteins (including enzymes) for oral administration are known in the art. Non-limiting examples of such methods include formulating the protein with enzyme inhibitors, such as chicken and duck ovomucoids and serine protease inhibitors; formulating proteins in mucoadhesive polymeric systems; formulating proteins in protective carrier systems such as emulsions, nanoparticles, microspheres, and liposomes; chemical modification of the protein with a moiety that makes it resistant to degradation or proteolysis, such as polyethylene glycol.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives.

An enzyme, nucleic acid, vector, and/or added agent can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Suitable carriers include saline solution and other materials commonly used in the art.

The compositions can also contain other ingredients such as other analgesics (e.g., acetaminophen, ibuprofen, or salicylic acid), vitamins, anti-microbial agents, or preservatives. It will be appreciated that the amount of an enzyme, nucleic acid, vector, or additional agent for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

### Nicotine-Degrading Enzymes

As noted above, the methods and compositions described herein use a nicotine-degrading enzyme or expression vector therefor.

Bacteria that can use nicotine as their sole carbon and nitrogen source were evaluated to identify efficient enzymes for nicotine degradation and therapeutic strategies. Strains belonging to Pseudomonas putida, a non-pathogenic member of the genus Pseudomonas, are thought to have a series of enzymes capable of metabolizing nicotine to fumaric acid.¹⁴

P. putida S16 was originally isolated from a soil sample from a field under continuous tobacco cropping in Shandong, People's republic of China.¹⁷ This S16 strain was found to be effective in degrading nicotine, and it has been shown that S16's metabolism of nicotine follows the pyrrolidine pathway.¹⁴ The enzyme found in the first committed step of S16's degradation of nicotine is NicA2 (PPS_4081), a flavin-containing enzyme. The amino acid sequence of this NicA2 protein is as follows (SEQ ID NO:1).

| | |
|---|---|
| 1 | MSDKTKTNEG FSRRSFIGSA AVVTAGVAGL GAIDAASATQ |
| 41 | KTNRASTVKG GFDYDVVVVG GGFAGATAAR ECGLQGYRTL |
| 61 | LLEARSRLGG RTFTSRFAGQ EIEFGGAWVH WLQPHVWAEM |
| 121 | QRYGLGVVED PLTNLDKTLI MYNDGSVESI SPDEFGKNIR |
| 161 | IAFEKLCHDA WEVFPRPHEP MFTERARELD KSSVLDRIKT |
| 201 | LGLSRLQQAQ INSYMALYAG ETTDKFGLPG VLKLFACGGW |
| 241 | NYDAFMDTET HYRIQGGTIG LINAMLTDSG AEVRMSVPVT |
| 281 | AVEQVNGGVK IKTDDDEIIT AGVVVMTVPL NTYKHIGFTP |
| 321 | ALSKGKQRFI KEGQLSKGAK LYVHVKQNLG RVFAFADEQQ |
| 361 | PLNWVQTHDY SDELGTILSI TIARKETIDV NDRDAVTREV |
| 401 | QKMFPGVEVL GTAAYDWTAD PFSLGAWAAY GVGQLSRLKD |
| 441 | LQAAEGRILF AGAETSNGWH ANIDGAVESG LRAGREVKQL |
| 481 | LS |

A nucleic acid that encodes the NicA2 enzyme with SEQ ID NO:1 is available as NCBI accession number CP002870.1 GI:338835784, where the SEQ ID NO:1 sequence is encoded at positions 4613081-4614529.

Although, NicA2 is an essential enzyme within the purview of P. putida's degradation of nicotine it naturally operates within a metabolic cascade. Hence, it was unclear if a single bacterial enzyme (isolated from the other enzymes in the metabolic cascade) would provide useful degradation of nicotine in vitro, or under mammalian in vivo conditions. As described herein, NicA2 is surprisingly effective at degrading nicotine under exactly the types of conditions that exist in subjects who smoke.

Biochemical evaluation of the NicA2 enzyme, including the determination of NicA2's Kₘ, k_{cat}, thermostability, half-life in buffer, serum, and in vivo as well as its product profile toxicity were performed and the results show that the NicA2 enzyme has utility for reducing and/or eliminating the toxicity associated with absorption of nicotine.

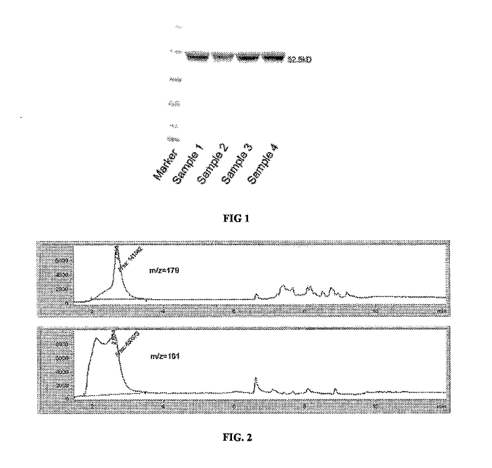

NicA2 was expressed in BL21(DE3) cells and purified by affinity chromatography. Under these conditions 21 mg/L of the 52.5 kDa NicA2 protein was obtained. Upon attaining pure NicA2 (FIG. 1), kinetics assays were initiated to determine catalytic parameters. However, unlike most enzymatic systems, the reaction of nicotine with NicA2 did not generate a single product. Instead, Nic2A enzymatic action on nicotine produced complex mixture of interconverting products.

As shown below, instead of generating cotinine, NicA2 has evolved so as to catalyze the oxidation of nicotine to N-methylmyosmine, 1.

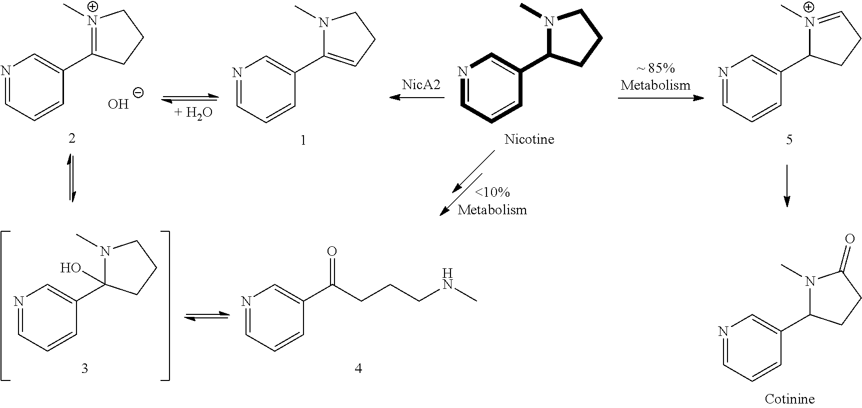

This 4,5 dihydropyrrole (1) can then undergo non-enzymatic ring tautomerism and hydrolysis to ultimately form pseudooxynicotine, 4.¹⁸ The tautomerism/hydrolysis of 1 occurs spontaneously and its equilibration is pH dependent.¹⁸ We observed three products by LC-MS, one with m/z 179 (4) and two inseparable nicotine metabolites with m/z 161 (1 and 2, see Examples and FIG. 2).

Due to the limitation of instrument sensitivity and the dynamic equilibration of the products, direct quantification of the enzyme's efficiency was challenging. However, the strategy that was ultimately successful involved integration to determine the areas of product peaks of 1, 2 and 4 and then back calculation to determine the amount of nicotine consumed by NicA2. This approach permitted accurate determination of the kinetic parameters of the enzyme.

As a result of the studies described in detail in the Examples, it was determined that NicA2 was very thermal stable and exhibited long-term stability at room temperature, at 37° C., and in mammalian serum.

One specific aspect of the invention is a method of degrading nicotine by contacting the nicotine with a NicA2 enzyme, to thereby degrade nicotine to N-methylmyosmine (1). Once this enzymatic step occurs, the N-methylmyosmine (1) compound hydrolyzes to form one or more non-toxic, non-addictive compounds. The NicA2 enzyme is highly stable in serum and is active (without loss of activity) at high temperatures (including 70° C.), and may have a half-life of about three days in mammalian serum in vitro. (The half-life of NicA2 in vivo in mammals is about 3-4 hours due to renal clearance.) Hence the NicA2 enzyme is useful in vivo for reducing nicotine toxicity and nicotine addiction.

Another specific aspect of the invention is a method involving administering at least one NicA2 enzyme, at least one expression vector encoding a NicA2 enzyme, or a composition of the NicA2 enzyme or the expression cassette, to a mammalian subject.

Thus, in accordance with the methods described herein nicotine-degrading enzymes can be used in compositions and methods for treating nicotine toxicity, nicotine addiction, promoting smoking cessation, reducing the relapse of nicotine consumption, or treating nicotine poisoning, as discussed above.

In some embodiments, the nicotine-degrading enzyme degrades nicotine into a non-addictive substance. In some embodiments, the nicotine-degrading enzyme degrades nicotine into N-methylmyosmine. In some embodiments, the nicotine-degrading enzyme degrades nicotine into 4-(methylamino)-1 (pyridine-3-yl)butan-1-one.

In specific embodiments, the nicotine-degrading enzyme is obtained from Pseudomonas putida. In further specific embodiments, the nicotine-degrading enzyme is NicA2. In further specific embodiments the nicotine-degrading enzyme has the amino acid sequence of SEQ ID NO:1.

In some embodiments, the nicotine-degrading enzyme is a NicA2 variant that exhibits nicotine-degrading activity in vivo. Variants of the nicotine-degrading enzyme can be employed in the compositions and methods described herein. For example, a nicotine-degrading enzyme can be modified or mutated to optimize the affinity, selectivity, activity, stability, half-life, or other desirable property of the nicotine-degrading enzyme. In general, variant or mutant nicotine-degrading enzymes have one or more of the amino acid residues that are different from what is present in the reference nicotine-degrading enzyme. Such variant and mutant nicotine-degrading enzymes necessarily have less than 100% sequence identity or similarity with the reference amino acid sequence. In some embodiments, a variant nicotine-degrading enzyme has at least 75%, 80%, 85%, 90% or 95% sequence identity with the amino acid sequence of the reference nicotine-degrading enzyme, such as NicA2. In specific embodiments, a NicA2 variant has at least 75%, 80%, 85%, 90% or 95% sequence identity with SEQ ID NO:1. In specific embodiments, the NicA2 variant has at least 80% or at least 95% sequence identity with SEQ ID NO:1. A variant nicotine-degrading enzyme can be screened for nicotine-degrading activity using any suitable in vitro or in vivo assay, such as illustrated in the examples below.

In specific embodiments, the amino acid sequence of the NicA2 variant is modified as compared to SEQ ID NO:1 to reduce immunogenicity in the subject. For example, potentially immunogenic epitopes can be identified, such as by using human T-cell based methods (such as EpiScreen), animal models (such as rodent immunogenicity models), and/or in silico predictions, and replaced with less immunogenic sequences. Additionally or alternatively, in specific embodiments, the amino acid sequence of the NicA2 variant is modified to enhance the catalytic efficiency of the enzyme. Additionally or alternatively, in specific embodiments, the amino acid sequence of the NicA2 variant is modified to enhance the stability of the enzyme.

Additionally, or alternatively, the nicotine-degrading enzyme may be modified to increase its half-life, such as by being conjugated or fused to a moiety that increases the circulating half-life of the nicotine-degrading enzyme in vivo. Methods for improving the pharmacokinetics of a peptide or protein, including increasing its circulating half-life, are known in the art. For example, the enzyme can be conjugated or fused to polyethylene glycol moieties, albumin moieties, or albumin-binding moieties. The enzyme can also be conjugated or fused to an antibody Fc domain or a peptide that mimics the half-life extending properties of polyethylene glycol. Thus, non-limiting examples of suitable moieties for increasing half-life include human serum albumin, polyethylene glycol, albumin-binding domains, albumin-binding peptides, transferrin, a constant domain (Fc fragment) of an immunoglobulin protein such as immunoglobulin G, a homo-amino acid polymer, a proline-alanine-serine polymer, an elastin-like peptide, and a negatively charged, highly sialylated peptide.

For example, by conjugating PEG moieties to a nicotine-degrading enzyme (such as NicA2), its residence time in the body can be increased and its degradation by proteolytic enzymes can be decreased. As noted above, PEGylation also may reduce immunogenicity. In particular, since the kidney generally filters out molecules below 60 kDa, PEG conjugation to a nicotine-degrading enzyme will increase its hydrodynamic radius so as to reduce kidney filtration. In addition, PEGylation may also increase the enzyme's solubility due to the hydrophilicity of PEG moieties and decrease the accessibility of the enzyme to degrading enzymes or antibodies. Currently, most PEGylated drugs on the market use linear PEG with sizes ranging from ˜5-20 kDa. Thus, in specific embodiments, linear or branched PEG moieties of such a size (such as PEG moieties having chain lengths between 5-20 kDa) are conjugated to the nicotine-degrading enzyme, such as via surface-exposed lysine residues available for conjugation. A wide range of methods for attaching PEG moieties to a protein are known. One example s via an activated monomethoxy-PEG ester, which can react with an amine(s) on the protein's surface.

Additionally or alternatively, the nicotine-degrading enzyme may be conjugated or fused to another protein that has an extended elimination half-life serum, such as an albumin moiety. Albumin (molecular mass ˜67 kDa) is the most abundant protein in plasma, present at 50 mg/ml (600 μM), and has an elimination half-life of 19 days in humans. Albumin's long elimination half-life is believed to be at least partly due to FcRn-mediated recycling following the same mechanism as IgG recycling. Thus, in some embodiments, the nicotine-degrading enzyme is conjugated or fused to an albumin moiety, such as a human serum albumin (HAS) moiety. Additionally or alternatively, the nicotine-degrading enzyme may be conjugated or fused to a small protein domain or peptide that binds albumin with high affinity.

In specific embodiments, any such modification increases the circulating half-life of the enzyme by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, or 72 hours, or longer. In further specific embodiments, the circulating half-life is increased by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days, or longer. In further specific embodiments, the half-life is extended such that the nicotine-degrading enzyme can be administered once weekly, twice monthly, once monthly, once every 6 weeks, once every two months, once every three months, once every six months, or less frequently, and still exhibit nicotine-degrading activity throughout the dosing interval.

### Expression Vectors

An expression cassette or expression vector that includes a nucleic acid segment encoding a polypeptide or peptide comprising a sequence with at least 95% sequence identity to any of SEQ ID NO:1 can be used to generate the NicA2 enzyme. For example, one of skill in the art can prepare an expression cassette or expression vector that can express one or more encoded the NicA2 enzymes. Host cells can be transformed by the expression cassette or expression vector, and the expressed polypeptides or peptides can be isolated therefrom. Some procedures for making such genetically modified host cells are described below.

The encoded the NicA2 enzymes can be operably linked to a promoter, which provides for expression of an mRNA encoding the B the NicA2 enzymes. The promoter can be a promoter functional in a host cell such as a viral promoter, a bacterial promoter or a mammalian promoter. The promoter can be a heterologous promoter. As used herein, “heterologous” when used in reference to a gene or nucleic acid refers to a gene or nucleic acid that has been manipulated in some way. For example, a heterologous promoter is a promoter that contains sequences that are not naturally linked to an associated coding region. Thus, a heterologous promoter is not the same as the natural NicA2 enzyme promoter.

NicA2 enzyme nucleic acids are operably linked to the promoter when so that the nucleic acid segment encoding the NicA2 enzyme is located downstream from the promoter. The operable combination of the promoter with the region encoding the NicA2 enzyme is a key part of the expression cassette or expression vector.

Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides a very low level of gene expression. An inducible promoter is a promoter that provides for the turning on and off of gene expression in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired. In some embodiments, the promoter is an inducible promoter and/or a tissue-specific promoter.

Examples of promoters that can be used include, but are not limited to, the T7 promoter (e.g., optionally with the lac operator), the CaMV 35S promoter (Odell et al., Nature. 313:810-812 (1985)), the CaMV 19S promoter (Lawton et al., Plant Molecular Biology. 9:315-324 (1987)), nos promoter (Ebert et al., Proc. Natl. Acad. Sci. USA. 84:5745-5749 (1987)), Adhl promoter (Walker et al., Proc. Natl. Acad. Sci. USA. 84:6624-6628 (1987)), sucrose synthase promoter (Yang et al., Proc. Natl. Acad. Sci. USA. 87:4144-4148 (1990)), α-tubulin promoter, ubiquitin promoter, actin promoter (Wang et al., Mol. Cell. Biol. 12:3399 (1992)), cab (Sullivan et al., Mol. Gen. Genet. 215:431 (1989)), PEPCase promoter (Hudspeth et al., Plant Molecular Biology. 12:579-589 (1989)), the CCR promoter (cinnamoyl CoA:NADP oxidoreductase, EC 1.2.1.44) isolated from Lollium perenne, (or a perennial ryegrass) and/or those associated with the R gene complex (Chandler et al., The Plant Cell. 1:1175-1183 (1989)).

Other constitutive or inducible promoters can be used with or without associated enhancer elements. Examples include a baculovirus derived promoter, the p10 promoter. Plant or yeast promoters can also be used.

Alternatively, novel tissue specific promoter sequences may be employed in the practice of the present invention. Coding regions from a particular cell type or tissue can be identified and the expression control elements of those coding regions can be identified using techniques available to those of skill in the art.

The nucleic acid encoding the NicA2 enzyme can be combined with the promoter by available methods to yield an expression cassette, for example, as described in Sambrook et al. (Molecular Cloning: A Laboratory Manual. Second Edition (Cold Spring Harbor, N.Y.: Cold Spring Harbor Press (1989); Molecular Cloning: A Laboratory Manual. Third Edition (Cold Spring Harbor, N.Y.: Cold Spring Harbor Press (2000)). For example, a plasmid containing a promoter such as the T7-lac promoter can be constructed or obtained from Snap Gene (see, e.g., website at snapgene.com/resources/plasmid_files/pet_and_duet_vectors_%28novagen%29/pET-43.1a%28+%29/). These and other plasmids are constructed to have multiple cloning sites having specificity for different restriction enzymes downstream from the promoter. The nucleic acid encoding the NicA2 enzyme can be subcloned downstream from the promoter using restriction enzymes and positioned to ensure that the DNA is inserted in proper orientation with respect to the promoter so that the DNA can be expressed as sense RNA.

Expression cassettes that include a promoter operably linked to the NicA2 enzyme coding region can include other elements such as a segment encoding 3′ nontranslated regulatory sequences, and restriction sites for insertion, removal and manipulation of segments of the expression cassettes. The 3′ nontranslated regulatory DNA sequences can act as a signal to terminate transcription and allow for the polyadenylation of the resultant mRNA. The 3′ nontranslated regulatory DNA sequence preferably includes from about 300 to 1,000 nucleotide base pairs and contains prokaryotic or eukaryotic transcriptional and translational termination sequences. Various 3′ elements that are available to those of skill in the art can be employed. These 3′ nontranslated regulatory sequences can be obtained as described in An (Methods in Enzymology. 153:292 (1987)). Many such 3′ nontranslated regulatory sequences are already present in plasmids available from commercial sources such as Clontech, Palo Alto, Calif. The 3′ nontranslated regulatory sequences can be operably linked to the 3′ terminus of the NicA2 enzyme coding region by available methods.

Once the nucleic acid encoding the NicA2 enzyme is operably linked to a promoter (e.g., and other selected elements), the expression cassette so formed can be subcloned into a plasmid or other vector (e.g., an expression vector). Such expression vectors can have a prokaryotic or eukaryotic replication origin, for example, to facilitate episomal replication in bacterial, vertebrate and/or yeast cells.

Examples of vectors that provide for easy selection, amplification, and transformation of the expression cassette in prokaryotic and eukaryotic cells include pET-43.1a(+), pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences include origins of replication to provide for autonomous replication of the vector, additional selectable marker genes, such as antibiotic or herbicide resistance, unique multiple cloning sites providing for multiple sites to insert DNA sequences, and/or sequences that enhance transformation of prokaryotic and eukaryotic cells.

In order to improve identification of transformed cells, a selectable or screenable marker gene can be employed in the expression cassette or expression vector. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., an antibiotic), or whether it is simply a trait that one can identify through observation or testing, i.e., by ‘screening.’ Many examples of suitable marker genes are known to the art and can be employed in the practice of the invention.

Included within the terms selectable or screenable “marker” genes are genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall.

Possible selectable markers for use in connection with the present invention include, but are not limited to, an ampicillin gene, which codes for the ampicillin antibiotic. Other examples include a neo gene (Potrykus et al., Mol. Gen. Genet. 199:183-188 (1985)) which codes for kanamycin resistance and can be selected for using kanamycin, G418, and the like; a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al., J. Biol. Chem. 263:12500-12508 (1988)); a dalapon dehalogenase gene that confers resistance to the herbicide dalapon; a mutated anthranilate synthase gene that confers resistance to 5-methyl tryptophan; a β-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Reports. 14:403 (1995).

The expression cassettes and/or expression vectors can be introduced into a recipient host cell to create a transformed cell by available methods. The frequency of occurrence of cells taking up exogenous (foreign) DNA can be low, and it is likely that not all recipient cells receiving DNA segments or sequences will result in a transformed cell wherein the DNA is stably integrated into the host cell chromosome and/or expressed. Some may show only initial and transient gene expression. However, cells from virtually any species can be stably transformed, and those cells can be utilized to generate antigenic polypeptides or peptides.

Transformation of the host cells with expression cassettes or expression vectors can be conducted by any one of a number of methods available to those of skill in the art. Examples are: transformation by direct DNA transfer into host cells by electroporation, direct DNA transfer into host cells by PEG precipitation, direct DNA transfer to plant cells by microprojectile bombardment, and calcium chloride/heat shock.

Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction.

Once the NicA2 enzyme expression cassette or vector has been constructed and introduced into a host cell, the host cells can be screened for the ability to express the encoded NicA2 enzyme by available methods. For example, the host cell media, or host cell extracts, can be tested for NicA2 enzyme activity. In another example, the NicA2 enzyme can be detected using antibodies that bind to the polypeptides or peptides. Nucleic acids encoding the NicA2 enzyme can also be detected by Southern blot, or nucleic acid amplification using complementary probes and/or primers.

The following statements are intended to describe and summarize various embodiments of the invention according to the foregoing description in the specification.
1. A method comprising administering at least one NicA2 enzyme, at least one expression vector encoding a NicA2 enzyme, or a composition of the NicA2 enzyme or the expression cassette, to a mammalian subject.
2. The method of statement 1, which degrades nicotine in the subject.
3. The method of statement 1 or 2, which reduces the concentration of nicotine in a subject, compared to a method where at least one NicA2 enzyme, or a composition thereof, is not administered to a subject.
4. The method of any of statements 1-3, which reduces the incidence of nicotine addiction in a subject, compared to a method where at least one NicA2 enzyme, or a composition thereof, is not administered to a subject.
5. The method of any of statements 1-4, which reduced the toxicity of nicotine in a subject, compared to a method where at least one NicA2 enzyme, or a composition thereof, is not administered to a subject.
6. The method of any of statements 1-5, wherein at least one NicA2 enzyme, expression cassette, or composition thereof is administered prior to intake of nicotine.
7. The method of any of statements 1-6, wherein at least one NicA2 enzyme, expression cassette, or composition thereof is administered during intake of nicotine.
8. The method of any of statements 1-7, comprising repeatedly administering the at least one NicA2 enzyme, expression cassette, or composition thereof to the subject.
9. The method of any of statements 1-8, comprising daily administering the at least one NicA2 enzyme, expression cassette, or composition thereof to the subject.
10. The method of any of statements 1-8, comprising twice or thrice weekly administering the at least one NicA2 enzyme, expression cassette, or composition thereof to the subject.
11. The method of any of statements 1-8, comprising weekly administering the at least one NicA2 enzyme, expression cassette, or composition thereof to the subject.
12. The method of any of statements 1-11, wherein the NicA2 enzyme is thermally stable.
13. The method of any of statements 1-12, wherein the NicA2 enzyme has an optimum temperature for activity of 70° C.
14. The method of any of statements 1-13, wherein the NicA2 enzyme has at least 95% sequence identity to an amino acid sequence with SEQ ID NO:1.
15. The method of any of statements 1-14, wherein the NicA2 enzyme has a Kₘ of 92 nM; a k_{cat} of (1.32±0.04)×10⁻² s⁻¹; a K_{cat}/Kₘ of 1.44×10⁵ s⁻¹·M⁻¹, or a combination thereof at 37° C.
16. The method of any of statements 1-15, where the NicA2 enzyme has a half-life of three days in mammalian serum.
17. The method of any of statements 1-16, where the NicA2 enzyme degrades nicotine to N-methylmyosmine (1).
18. A composition comprising at least one NicA2 enzyme, or an expression vector encoding a NicA2 enzyme.
19. The composition of statement 18, further comprising a carrier.
20. The composition of statement 18 or 19, comprising a pharmaceutically acceptable carrier.
21. The composition of any of statements 18-20, formulated for parenteral administration.
22. The composition of any of statements 18-21, formulated for oral administration.
23. The composition of any of statements 18-22, comprising a therapeutically effective amount of the NicA2 enzyme or the expression vector.
24. The composition of statement 23, where the therapeutically effective amount is sufficient to degrade at least 500 nanomolar nicotine, or at least 1-300 nM or 5-60 ng/ml nicotine in serum.
25. The composition of statement 23 or 24, where the therapeutically effective amount is sufficient to degrade at least 500 nM nicotine to N-methylmyosmine (1), which hydrolyzes to a non-toxic and/or non-addictive compound.
26. The compositions of any of statements 23-24, where the therapeutically effective amount is sufficient to degrade at least 500 nM nicotine to N-methylmyosmine (1), which hydrolyzes to 4-(methylamino)-1-(pyridine-3-yl) butan-1-one (4).
27. The compositions of any of statements 18-26, comprising at least 20 nanomolar NicA2 enzyme, or an amount effective to achieve a serum concentration of enzyme of from about 0.1 μM to about 50 μM, from about 0.5 μM to about 10 μM, or about 4 μM.

The following non-limiting Examples illustrate certain aspects of the invention.

### Example 1: Materials and Methods

This Example describes some of the materials and methods employed in developing the invention.

### Materials

The plasmid containing NicA2 (PPS_4081) gene was a gift from Prof. Ping Xu (Shanghai Jiaotong University, China). The E. coli strain for plasmid amplification was MAX Efficiency® DH5α™ Competent Cells from Life Technologies. Amplified plasmids were purified using QIAprep Spin Miniprep Kit from QIAGEN. The E. coli cells for expression were BL21-CodonPlus (DE3)-RIL Competent Cells from Agilent Technologies. (S)-(−)-nicotine was purchased from Alfa Aesar (USA). The internal standard, nicotine methyl-D3 was purchased from Cambridge Isotope Laboratories. 4-(methylnitro-samino)-1-(3-pyridyl)-1-butanone (NNK) was purchased from Sigma-Aldrich (USA). All other chemical reagents, unless otherwise specified, were purchased from Sigma-Aldrich.

### NicA2 Expression and Purification

The plasmid containing NicA2 gene was obtained from Shanghai Jiaotong University, China and transformed into the E. coli strain DH5α cells for amplification, and then to E. coli. BL21(DE3) strain for expression. The E. coli. BL21 was cultured in LB medium at 37° C. until OD600 reached 0.8. IPTG (Isopropyl β-D-1-thiogalacto-pyranoside) was added at 1 mM to induce NicA2 expression. The culture was transferred at 16° C. and incubated overnight. The cells were harvested, lysed and NicA2 was purified with TALON metal affinity resin. Pure NicA2 was dialyzed in PBS and confirmed by SDS-PAGE (FIG. 1). The enzyme solution was concentrated by Amicon® Ultra centrifugal filter devices (10 kD), concentration determined by BCA assay kit (Pierce™) and stored at 4° C.

### LC-MS for NicA2 Activity Assay

NicA2 activity was determined by LC-MS using Agilent 1260 Infinity liquid chromatography system with 6130 quadrupole mass spectrometry. 20 μl of each sample was injected to a Poroshell 120 EC-C8 column (4.6×50 mm, 2.7 μm, Agilent Technologies) subjected to a gradient (A to B where A=0.1% formic acid in water and B=0.1% formic acid in acetonitrile) of 0% B for 3 min, 0% B to 100% B from 3 to 7 min, and 100% B from 7 to 10 min at a constant flow rate of 0.5 ml/min. A column-solvent equilibration time of 3 min was conducted prior to next sample analysis. MS operational parameters were: API-ES mode, channel 1 (90%) positive single ion monitoring (SIM) of m/z 179 (30%), 161 (30%), 166 (30%) and 163 (10%), corresponding to the M+ peak of the reaction products, labeled internal standard and substrate respectively and channel 2 (10%) scan for positive ions; nitrogen as a nebulizing and drying gas (35 psi, 12 L/min), HV capillary voltage at 4 kV and the drying gas temperature to 300° C. To protect the detector from salts in the buffer, MS was turned on with a delay 1.4 min after injection.

### NicA2 Michaelis-Menten Assay

Nicotine was solubilized in ddH2O to a concentration of 10 mM as stock and diluted with HEPES buffer (50 mM, pH=7.4) in the assay. Then 100 μL nicotine solution was mixed with 100 μL NicA2 solution to obtain final concentrations of 0.0625, 0.125, 0.25, 0.5, 1, 2 nicotine and 10 nM NicA2. After incubating at room temperature for 20 min, 20 μL nicotine methyl D-3 (2 μM in 20% TFA/H2O) solution was added to the mixture as an internal standard and to quench the reaction as well. The samples were injected into the LC-MS for analysis.

For standard curve generation, 0.03125, 0.0625, 0.125, 0.25, 0.5, 1, 2 μM nicotine and 0.4 μM NicA2 was tested and incubated for 2 h for complete oxidation of nicotine.

For the assay at 37° C., all the operations were undertaken in a warm room (37° C.) and solutions were pre-warmed to 37° C. Subsequently protocols were followed as described, vide supra.

### NicA2 Stability Assay

Four μM nicotine and 40 nM NicA2 were prepared in HEPES buffer and stored at 37° C. Upon sitting for 0, 1, 3, 4, 5, 8, 12, 16, 20, or 25 days, 100 μL of each solution was extracted, mixed, incubated, quenched, analyzed as detailed, vide supra.

For serum stability assessment a more robust signal to noise ratio was needed. To obtain this parameter 40 μM nicotine and 400 nM NicA2 were made as stock solutions and stored at 37° C. At the time points of 0, 30 minutes, 1 hour, 3 hours, 2 days, 3 days, 4 days or 5 days, 50 μL of each solution was mixed and incubated for 20 min. The reaction was quenched and the protein was precipitated with 400 acetonitrile. The samples were centrifuged at 10000 rpm for 15 minutes and 400 μL of the supernatant from each sample was transferred to a new tube and evaporated. Finally, 200 μL of HEPES buffer and 20 μL nicotine methyl D-3/TFA was added to each sample for LC-MS analysis.

### Temperature Curve of NicA2

Four μM nicotine and 40 nM NicA2 were prepared in HEPES buffer and pre-incubated at 20, 25, 30, 35, 40, 45, 50, 55, 60, 70, 75, 80, 90 or 95° C. for 1 minute in Eppendorf Mastercycler® personal. 100 μL of each solution was mixed, incubated, quenched and then analyzed as detailed, vide supra.

### NicA2 Co-Factor Assay

An FAD or FMN stock solution was added to nicotine HEPES solution to obtain 80 μM FAD or FMN mixed with 4 μM nicotine. 100 μL each sample was mixed with

100 μL 40 nM NicA2 and incubated at room temperature for 20 min. An additional sample without FAD or FMN was prepared at the same time as the control. The samples were analyzed as described above.

### NNK Toxicity to Mice

Short-term experiments were performed by administrating 35, 70 140 ng of NNK to male Swiss Webster mice (about 20 g, n=4 for each group) daily. Their viability and behavior was recorded.

Long-term experiments were performed by administrating 200 ng to the same species of mice (n=7) every other day for 5 weeks. At the end of the 5^{th} week, the mice were euthanized and autopsied.

### Data Analysis

Raw data was obtained with Agilent ChemStation, ions with m/z 179,161 (products), 166 (internal standard), 163 (nicotine) were extracted from MS channel 1 (SIM). The area of each peak was integrated and divided by internal standard (IS).

Data was further analyzed with Prism 6.0. For standard curves, the ratios of products to IS were plotted against concentrations of converted nicotine and fitted with linear regression. For experiment data, the ratios were fitted to the standard curves to obtain converted nicotine concentration, and then divided by reaction time (20 min) to get v0. These v0 were plotted against substrate nicotine concentrations using he Michaelis-Menten model to obtain Km and Vmax.

### Example 2: Enzymatic Breakdown of Nicotine by NicA2

As shown below, NicA2 has evolved so as to catalyze the oxidation of nicotine to N-methylmyosmine, 1.

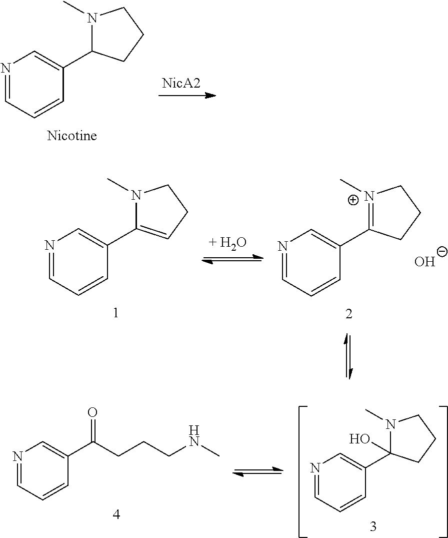

This 4,5 dihydropyrrole (1) can then undergo non-enzymatic ring tautomerism and hydrolysis to form pseudooxynicotine, 4. The tautomerism/hydrolysis of 1 occurs spontaneously and its equilibration is pH dependent.

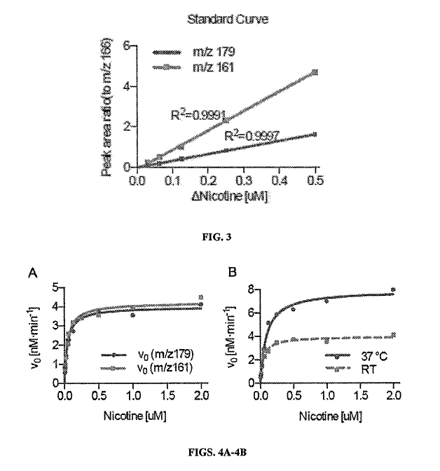

Upon reaction of nicotine with NicA2, three products were observed by LC-MS, one with m/z 179 (4) and two inseparable nicotine metabolites with m/z 161 (1 and 2, FIG. 2). Due to the limitation of instrument sensitivity and the dynamic equilibration of the products, direct quantification of the enzyme's efficiency was challenging. To compensate for these shortcomings, the product peaks 1, 2 and 4 were integrated in order to back-calculate the amount of nicotine consumed by NicA2. Standard curves were generated wherein nicotine was fully oxidized by NicA2 and the m/z 161/179 signals were plotted against a change in nicotine concentration, to provide a direct linear relationship (FIG. 3). The kinetic parameters of the enzyme were thus accurately determined.

To determine kinetic parameters of the enzyme, curves were generated at varying nicotine concentrations utilizing 10 nM NicA2 at room temperature. Samples were analyzed by LC-MS using nicotine (methyl-D3) as an internal standard. Target m/z values (161, 179 and 166) were extracted, integrated and fit to obtain the velocity, v₀. The v₀ was plotted against a series of nicotine concentrations and these data were fit to the Michaelis-Menten equation (FIG. 4A). The Kₘ and k_{cat} values for NicA2 were derived from integrating the two different m/z peaks shown in Table 1.

TABLE 1
Michaelis-Menten parameters of NicA2 at room temperature

| | m/z 179 | m/z 161 |
|---|---|---|
| Kₘ [nM] | 43.5 ± 4.7 | 46.2 ± 6.4 |
| k_{cat} [s⁻¹] | (6.64 ± 0.17) × 10⁻³ | (7.02 ± 0.23) × 10⁻³ |
| k_{cat}/Kₘ | 1.53 × 10⁵ | 1.52 × 10⁵ |
| [s⁻¹ · M⁻¹] | | |

The Kₘ, k_{cat} and k_{cat}/Kₘ values in Table 1 are almost identical whether they were derived using the m/z 161 peak or the m/z 179 peak. These results indicate that the methods employed are accurate. Because the m/z 179 was a single peak and was integrated more accurately, the results from m/z 179 were used for further studies.

### Example 3: Stability of NicA2

Enzyme activity can be highly sensitive to temperature. For example, cocaine bacterial esterase CocE has a half-life of 11 min in aqueous milieu¹¹ and 13 minutes in serum.¹⁹ As the temperature increases, the expected increase in velocity resulting from increased enzyme-substrate collisions can be offset by denaturation.

At room temperature, NicA2 showed excellent activity. To evaluate whether NicA2 would also have good activity in vivo, the same assay was run at 37° C. As anticipated at this temperature, both Kₘ and k_{cat} were increased. Significantly, the specificity constant k_{cat}/Kₘ remained virtually unchanged (FIG. 4B).

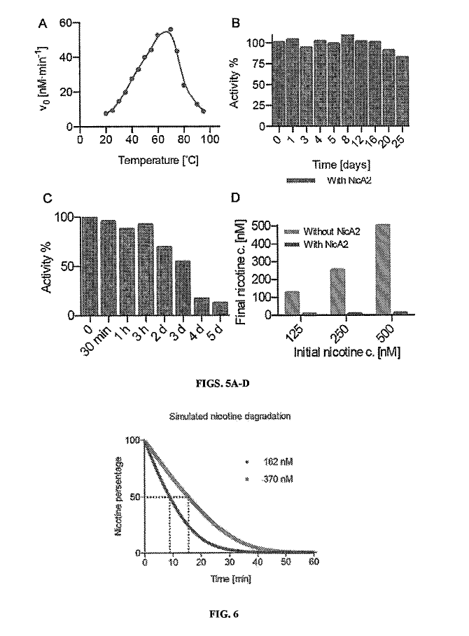

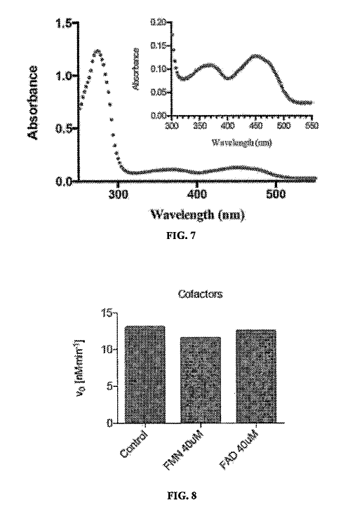

The effect of higher temperatures on the enzyme's stability was also examined. Surprisingly, the NicA2 enzyme has an “optimum temperature” of 70° C., indicating that the enzyme is remarkably thermally stable (FIG. 5A).

To be a candidate for nicotine addiction therapy, the enzyme should possess longevity in buffer and ideally in serum. To test for these metrics, NicA2 was incubated at 37° C. in HEPES buffer and enzyme activity was examined at different time points. Again, unforeseen, yet impressively the enzyme showed excellent stability and activity over 3 weeks (FIG. 5B) and a half-life of 3 days in mice serum (FIG. 5C). Hence, NicA2 is much more stable than the cocaine bacterial esterase CocE, with its half-life of only 11 min in aqueous milieu and 13 minutes in serum.

### Example 4: Reducing the Effects of Smoking by Use of NicA2

Smoking one cigarette provides an absorbed nicotine dose of about 1-2 mgs and results in a peak concentration of 20-60 ng/ml (162-370 nM) in blood.¹² The results provided herein reveal that NicA2 has a Kₘ of 43 nM (92 nM at 37° C.), which is well below the concentration range of nicotine in serum. In theory this would equate to the enzyme working at saturating conditions.

As a means to test NicA2's efficiency “in vivo”, nicotine was doped with or without enzyme in serum (FIG. 5D). The enzyme in a 30-minute window consumed all nicotine whereas in the background reaction nicotine remained fully stable. To further substantiate a conclusion that NicA2 can remove nicotine in vivo, NicA2's catabolism of nicotine was simulated based upon foregoing kinetic constants that were determined. The results of this simulation are shown in FIG. 6. NicA2 specificity constant is approximately 10⁵ M⁻¹s⁻¹ and while clearly far from a perfect enzyme (10⁸-10⁹ M⁻¹s⁻¹). NicA2 at 20 nM still possesses enough “catalytic power” to readily decrease nicotine's half-life from 2-3 hours for a single cigarette to 9-15 minutes, (5.0 mg NicA2 for a 70 kg person).

### Example 5: NicA2 Cofactors

This Example provides evidence that NicA2 has a cofactor that is covalently or tightly bound to NicA2.

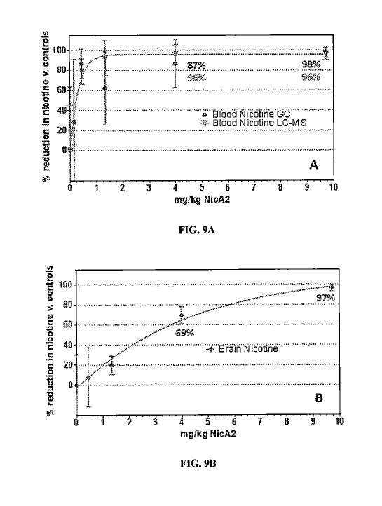

The UV-vis spectrum of NicA2 is consistent with the presence of a flavin cofactor associated with NicA2 (FIG. 7), because the minor peak at 370 nm and the major peak at 450 nm match are typically observed for flavin proteins. Moreover, the yellow tint associated with flavin co-purified with NicA2. In addition, FIG. 8 shows that when an excess of FMN or FAD was added to a reaction mixture of NicA2 and nicotine, there was no change in NicA2 activity. These data indicate that the flavin is either covalently or tightly bound, which is a useful property for a therapeutic, because co-administration of a flavin cofactor will not be necessary.

### Example 6: Reaction Products

The clinical utility of an enzyme also relates to the toxicity and addictive properties of the reaction products. Pseudooxynicotine (4) has not been reported to possess addictive properties. However, it has been reported as a likely precursor to 4-(methylnitrosamino)-1-(3-pyridyl)-1-butanone (NNK), a potential carcinogen.²⁰ To investigate any harmful effects of 4, toxicology studies were performed in which mice were administered pseudooxynicotine (4).

Mice received 35, 70 or 140 ng of compound 4 daily, which is representative of the typical range of nicotine amounts (4-72 ng/mL) found in smokers,¹⁹ if fully converted to compound 4. After five days of such administration, none of the mice exhibited any evidence of health or behavioral problems at any of the listed dosages. In addition, the long-term exposure (5-weeks) of 4 was evaluated where the mice were dosing every other day with 200 ng compound 4 per dose. All mice at either dosing regimen remained healthy and autopsies did not reveal any sign of neoplasia or organ damage.

These data indicate that the enzyme degradation products of nicotine do not incur safety issues.

### Example 7: In Vitro NicA2 Activity at Physiologically Relevant Nicotine Levels

NicA2 activity studies conducted in rat serum at nicotine levels spanning those observed in smokers at clinically-feasible enzyme concentrations (20-400 nM) demonstrated substantial reduction in nicotine levels at 4 min in a nicotine and enzyme concentration-, and time-dependent manner. NicA2 was diluted in rat serum (controls: serum without NicA2) to give a final concentration of 400 nM, 200 nM, and 20 nM. Nicotine was diluted in 50 mM HEPES pH 7.5 to give final amounts of 640, 360, 80, 40, 20, 10, 5 and 2.5 ng (spanning a nicotine concentration range of 3.2 μg/ml to 12.5 ng/ml). The diluted NicA2 in serum and diluted nicotine were mixed 1:1 after pre-equilibration at 37° C. and 5 min (in 200 μl total). After 4 minutes or 20 minutes, reactions were diluted and quenched by addition of 800 μl H₂O+500 μl 2M NaOH/0.2M NH₄OH. At the highest NicA2 concentration (400 nM) initial plasma nicotine levels of 25 and 50 ng/ml dropped to <2 ng/ml at 4 min. (Table A).

TABLE A
% Reduction in nicotine levels in vitro

| NicA2 | Nicotine Conc. | | |
|---|---|---|---|
| conc. | 25 ng/ml | 50 ng/ml | 100 ng/ml |
| 400 nM | 100% | 100% | 94% |
| 200 nM | 100% | 100% | 72% |
| 20 nM | 17% | 8% | 4% |

### Example 8: In Vivo Testing of NicA2

Rats were used for in vivo testing because their nicotine metabolism is generally similar to that of humans with regard to rate and range of metabolites. 30 female, 250 g Sprague Dawley rats were pretreated with varying amounts of NicA2 intravenously as noted in Table B below.

TABLE B

| | Number of | | |
|---|---|---|---|
| Group | Animals | NicA2 | |
| 1 | 5 | 0 | mg/kg |
| 2 | 5 | 4 | mg/kg |
| 3 | 5 | 1.33 | mg/kg |
| 4 | 5 | 0.44 | mg/kg |
| 5 | 5 | 0.15 | mg/kg |
| 6 | 5 | 9.72 | mg/kg |

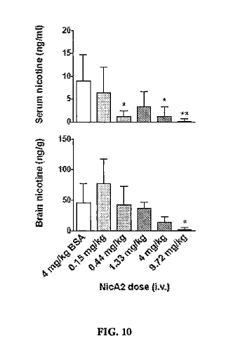

After administration of NicA2 (generally over 5 minutes), nicotine (0.03 mg/kg) was administered intravenously. At 5 minutes, the rats were sacrificed under isoflurane anesthesia and 1 ml blood samples were collected with 0.32 ml heparin. 0.5 ml of each blood sample was added to a tube containing ice cold 2 ml methanol (to quench NicA2 activity) and the contents were rapidly mixed. Another 0.5 ml of each blood sample was added to a tube containing ice cold 2 ml of methanol (to quench NicA2 activity) and deuterium labeled D4-nicotine, and the contents were rapidly mixed. The brains were collected and frozen at −20° C. Results are shown in FIGS. 9A-9B and 10. Measurements were made in accordance with Keyler et al., International Pharmacology 2008, 8, 1589-1584, and, Hieda et al., Psychopharmacology, 1999, 143(2), 150-157.

As shown in the figures, pretreatment with a range of NicA2 doses showed robust enzymatic activity with the highest dose leading to a >95% reduction in nicotine blood and brain levels measured 5 min after an i.v. bolus dose of 0.03 mg/kg nicotine compared to controls. FIG. 9A shows the %-reduction (mean, SD) in blood nicotine levels, with results for both the Gas Chromatography (GC) and LC-MS1 methods reported and showing similar results. FIG. 9B depicts the %-reduction in brain nicotine levels. Notably, 4 out of the 5 mice had brain nicotine levels below the detectable limit of 2 ng/ml of nicotine. The data indicate that a higher NicA2 dose (such as about 10 mg/kg) may be needed to achieve a greater than 95% reduction of nicotine in the brain than in serum.

These results are notable as they show that the unmodified, un-stabilized, wild-type NicA2 enzyme leads to near elimination of nicotine in blood and brain at clinically-feasible NicA2 doses, even following a rapid-loading (10 s) bolus i.v. injection of nicotine equivalent to 2 cigarettes (on a mg/kg basis) compared to the 7-10 minutes it takes to smoke one cigarette. As a first approximation, the highest 9.72 mg/kg NicA2 dose tested in rats would translate, based on an established allometric scaling algorithm (See, e.g., West et al., Journal of Experimental Biology, 2005, 208(9), 1575-1592), to <200 mg dose in a 70 kg human (2.9 mg in 300 mg rat).

### Example 9: Pharmacokinetics (PK) of Enzyme Effects on Nicotine Disposition

Kinetic screening is conducted in rat serum for NicA2 enzyme activity and stability in vitro. Initial screening is conducted using established assay protocols and detection methods, such as those described, for example, in Xue, S et al., J Am Chem Soc 2015, 137(32), 10136-10139, and Hieda, Y. et al., Psychopharmacology 1999, 143(2), 150-157), and illustrated above.

The enzyme kinetics is tested and confirmed in vivo using rats, since the rate of nicotine elimination and identity of metabolites generated in rats are similar to those in humans.

### i) Single Dose Nicotine-PK.

Groups of 8 male and female Holtzman rats, with group size powered to detect a 75% reduction in brain nicotine concentration are pre-treated with NicA2 enzyme followed in 5 minutes by nicotine (30 μg/kg) dosed intravenously. This nicotine dose produces serum nicotine levels within the range measured in smokers (20-30 ng/ml). The NicA2 dose is adjusted based on its in vitro activity but initially brackets 4.0 mg/kg based on the initial in vivo data discussed above. Blood is sampled at 1, 3, and 5 minutes after nicotine dosing to establish its onset of action. Animals are sacrificed at 5 minutes, blood and brain collected and immediately processed to quench NicA2 activity by addition of methanol.

Nicotine levels are measured by GC or LC-MS. Blood rather than serum nicotine levels are measured to allow rapid addition of methanol to quench the samples (which causes hemolysis). Brain is processed similarly, and brain nicotine levels is corrected for the blood content of brains.

Groups of treated rats are compared to controls receiving bovine serum albumin (BSA) to assess % reduction in blood and brain nicotine concentration. Data is analyzed using t-tests for 2 groups or ANOVA for multiple groups with Bonferroni's post-test.

### ii) NicA2 PK and Duration of Action.

His6-tagged NicA2 is dosed intravenously at 5 mg/kg. For NicA2, blood-sampling is done at pre-dose and then over a 0.25-120 hour period with 8 animals per time-point. For studies on NicA2 variants with extended half-lives, sampling is extended to 4-5× the expected elimination-t′/2 of the variant. Quantification of His6-tagged NicA2 in serum is performed through an ELISA method using rabbit anti-NicA2 IgG for detection.

To confirm whether detected NicA2 is still active at the time of sampling, residual enzyme activity in the serum samples is assessed by conducting an ex vivo activity assay. Serum samples are spiked with a known amount of nicotine, and decreases in nicotine and/or increases in product formation are measured relative to test samples containing known amounts of added fresh NicA2. This permits the assessment of residual NicA2 activity after circulating in the animal at each timepoint.

### Example 10: Effects of Nicotine-Degrading Enzyme on Nicotine Disposition in Rats: in-Depth Studies

The effects of nicotine-degrading enzyme over a range of clinically-relevant enzyme doses and routes of administration and single and repeated nicotine doses are studied to confirm the ability of the enzyme(s) to alter nicotine distribution and metabolism, using assays that are well understood in the art, such as those described in Keyler, D. et al., International Pharmacology 2008, 8, 1589-1594, and Pentel, P. et al., Adv. Pharmacol. 2014, 69, 553-580). For convenience, protocols are described below with reference to NicA2, but other nicotine-degrading enzymes, including NicA2 variants, will be used.

### i) Dose-Response Relationships.

Dose-response relationships are assessed using an experimental design similar to that of Example 9, with NicA2 pretreatment followed by nicotine dosing and sampling at 5 minutes in 2 separate experiments: (a) over a range of NicA2 doses (0, 0.5, 1.5, 4.5 mg/kg) using a fixed 30 μg/kg nicotine dose, with groups compared to the 0 μg/kg controls and (b) over a range of nicotine doses (15, 30, 60 μg/kg) using a fixed NicA2 dose chosen on the basis of (a), and with each group compared to a control receiving the same dose of nicotine without NicA2.

### ii) Repeated Nicotine Doses.

The effects of repeated nicotine doses is assessed by a protocol wherein NicA2 administration is followed by repeated i.v. doses of nicotine (30 μg/kg) every 14 minutes for 16 hours totaling 1 mg/kg, a well-studied paradigm which produces serum nicotine levels typical of chronic smokers (see, e.g., Pentel, P. et al., JPET 2006, 317, 660-666) and rats self-administering nicotine (see, e.g., LeSage, M. et al., Psychopharmacology 2006, 409-416). Rats receiving NicA2 prior to nicotine dosing are compared to controls receiving saline. Blood nicotine levels are sampled periodically and brain is sampled at the end.

iii) Route of Administration.

The i.v., subcutaneous (s.c.), and intramuscular (i.m.) routes for dosing NicA2 v. controls are studied and serum and brain nicotine levels are measured in separate groups when nicotine is administered 2, 5 or 15 minutes after NicA2 administration by different routes.

### Example 11: Nicotine Discrimination Testing

Drug discrimination is a common method for indicating medication efficacy since it models the acute subjective effects that drug abusers feel when they take a single dose of a drug, presumably pleasant/euphoric effects. The discrimination assay is a useful initial behavioral screen as it is sensitive to addiction treatments such nicotine-specific mAb (see, e.g., LeSage, M. et al., Pharmacology, biochemistry, and behavior 2012, 102, 157-62), and animals can be maintained on the procedure for over a year, allowing repeated testing of multiple enzyme designs and/or doses in the same animal, if needed. Thus, the discrimination model can facilitate dose-finding for enzyme efficacy in a behavioral setting and avoid proceeding to the more time-consuming and costly self-administration studies with an ineffective enzyme dose. For convenience, protocols are described below with reference to NicA2, but other nicotine-degrading enzymes, including NicA2 variants, will be used.

Two groups (NicA2 and control) of n=8 rats are trained to discriminate nicotine from saline using methods known in the art. In standard operant conditioning chambers during daily 15-min sessions, rats are initially trained to press one lever for food pellets following a subcutaneous injection of 0.4 mg/kg nicotine and press the opposite lever following saline.

Discrimination is considered stable when (a) discrimination criteria are met during two consecutive saline and nicotine test sessions, (b) >95% injection-appropriate responding is exhibited on six consecutive training sessions, and (c) response rates are stable. Then the NicA2 or control is administered after the final training session. The following four consecutive sessions are nicotine test sessions as described above to assess the timecourse of NicA2 effects.

The percentage of responding on the nicotine-appropriate lever (% NLR) across the four test sessions is the primary dependent variable, comparing between groups using two-factor ANOVA with Bonferroni-corrected t-tests for post-hoc pairwise comparisons.

### Example 12: Nicotine Self-Administration Testing

The ability of the enzyme to block the reacquisition of nicotine self-administration (NSA) is studied as potentially relevant to showing efficacy in relapse prevention. For convenience, protocols are described below with reference to NicA2, but other nicotine-degrading enzymes, including NicA2 variants, will be used.

Two groups (NicA2 and control) of 10 rats are implanted with jugular cannulas one week after arrival. One week later the rats are placed in operant cages and allowed to acquire NSA using 23 hour sessions and a nicotine unit dose of 0.03 mg/kg and an escalating fixed-ratio (FR) schedule of 1, 2 and 3 at weekly intervals. Total nicotine intake in control rats averages ˜1.5 mg/kg/d or the equivalent of 2-3 packs of cigarettes/day, providing a robust test of NicA2 efficacy.

Responses on the active lever are compared to the inactive lever to confirm that rats are responding for nicotine (active:inactive ratio >2:1). After at least one week at FR 3 and when NSA is stable (<15% variation and no trend), extinction is arranged by substituting saline for nicotine. After ≥1 week, when active lever pressing has decreased by ≥60%, and no trend in active leverpressing is observed, NicA2 is administered and rats are given access to the nicotine training dose to allow reacquisition of NSA for 10 days. Rats receive additional doses of NicA2 during the reacquisition phase as needed, based on the enzyme's PK.

Mean NSA rates over these 10 days of reacquisition are compared by two-way ANOVA with Bonferroni-corrected t-tests for post-hoc pairwise comparisons to determine whether the enzyme blocks or reduces NSA reacquisition.

### Example 13: NicA2 PK Study

In order to assess conjugation to PEG or fusion to an albumin binding domain (ABD) to improve pharmacokinetic properties of NicA2, a PK study in mice was conducted. Purified His-tagged NicA2 was conjugated to 20 kDa PEG and subsequently purified by HPLC. NicA2-ABD fusion protein was generated by fusing an albumin binding domain (the ABD035 variant disclosed in Jonsson, et al., Protein Engineering, Design & Selection 2008, 21(8), 515-527) to the C-terminus of NicA2 through a flexible peptide linker followed by a C-terminal His₆-tag. The fusion protein was expressed in E. coli and purified as described above for NicA2.

His-tagged NicA2, NicA2-PEG or NicA2-ABD was dosed intravenously in mice at 5 mg/kg. Blood sampling was done at pre-dose and then over a 5 min-72 h period (three animals per time-point) and processed to serum.

Assay of serum samples was done taking advantage of the C-terminal His-tag on the test articles. MaxiSorp ELISA plates (Nunc) were coated overnight with anti-His×6 antibody (purchased from R&D Systems). Plates were blocked with 4% dry milk (purchased from Bio-Rad) in PBS. Dilutions of standard and serum samples in 2% milk in PBS+0.1% Tween-20 were added to the plates, and incubated for 1 hour at room temperature. After washing away unbound substances (all wash steps performed in PBS+0.1% Tween-20), rabbit anti-NicA2 polyclonal primary detection antibody (purchased from Noble Life Sciences, Inc.) was added to the wells for a 1 hour incubation. A wash step was followed by addition of horseradish peroxidase (HRP)-conjugated goat anti-rabbit IgG (purchased from Jackson ImmunoResearch). Plates were washed, and the remaining binding complex was detected with TMB substrate (3,3′,5,5′-tetramethylbenzidine; KPL). Once stopped with acid, plates were read on a spectrophotometer at 450 nm and data analyzed in SoftMax® Pro, version 6.5.1 (purchased from Molecular Devices). Concentrations in serum samples were obtained by extrapolation from the standard curves generated with the relevant molecule, and log(Concentration) was plotted as a function of time.

The slopes of the graphed lined obtained by linear regression of the data (GraphPad Prizm 6) were used to calculate the half-lives (see Table 2 below):

TABLE 2

| Protein | Half-life (h) | Fold Improvement |
|---|---|---|
| NicA2 | 3.7 | — |
| NicA2-PEG | 10.7 | 3 |
| NicA2-ABD | 40.5 | 11 |

The results show that both conjugation to PEG and fusion to an albumin binding domain significantly reduced the clearance of NicA2. The half-life of endogenous mouse serum albumin has been reported to be 35-40 hours (see, e.g., Chaudhury et al., J. Exp. Med. 2003, 197, 315-322). The NicA2-ABD fusion consequently achieved the longest theoretical possible half-life in mice. As the ABD is cross-reactive with human albumin, a substantial half-life in expected in humans; potentially approaching the 19 day half-life reported for human serum albumin. It is expected that making similar modifications to other nicotine-degrading enzymes, including NicA2 variants, also would prolong half-life.

### REFERENCES

(1) World Health Organization. WHO Report on the Global Epidemic, 2013: Enforcing Bans on Tobacco Advertising, Promotion, Sponsorship; WHO Press: Geneva, Switzerland, 2013.
(2) McGinnis, J. M.; Foege, W. H., JAMA 1993, 270, 2207-12.
(3) Stead, L. F.; Perera, R., et al., Cochrane Db Syst Rev 2008, 1, 1-163.
(4) Piper, M. E.; Federman, E. B., et al., Nicotine Tob Res. 2007, 9, 947-54.
(5) Koegelenberg, C. F.; Noor, F., et al., JAMA 2014, 312, 155-61.
(6) Hatsukami, D.; McBride, C., et al., J. Subst. Abuse 1991, 3, 427-40.
(7) (a) Goniewicz, M. L.; Delijewski, M., Hum. Vaccin. Immunother. 2013, 9, 13-25; (b) Lockner, J. W.; Lively, J. M., et al., J. Med. Chem. 2015, 58, 1005-1011; (c) Sliwinska-Mosson, M.; Zielen, I., et al., Acta Pol. Pharm. 2014, 71, 525-30; (d) Moreno, A. Y.; Azar, M. R., et al., Mol. Pharm. 2010, 7, 431-41; (e) Lockner, J. W.; Ho, S. O., et al., Bioorg. Med. Chem. Lett. 2013, 23, 975-8; (f) Isomura, S.; Wirsching, P., et al., J Org. Chem. 2001, 66, 4115-21; (g) Meijler, M. M.; Matsushita, M., et al., J Am. Chem. Soc. 2003, 125, 7164-5; (h) Moreno, A. Y.; Azar, M. R., et al., Vaccine 2012, 30, 6665-70.
(8) Wolters, A.; de Wert, G., et al., Addiction 2014, 109, 1268-73.
(9) Hoogsteder, P. H. J.; Kotz, D., et al., Addiction 2014, 109, 1252-1259.
(10) (a) Nabi Biopharmaceuticals, 2007, (b) Pentel, P. R.; LeSage, M. G., Adv. Pharmacol. 2014, 69, 553-80.
(11) Gao, D.; Narasimhan, D. L., et al., Mol. Pharmacol. 2009, 75, 318-23.
(12) Benowitz, N. L.; Hukkanen, J., et al., Handb. Exp. Pharmacol. 2009, 29-60.
(13) Benowitz, N. L., NIDA Res. Monogr. 1990, 99, 12-29.
(14) Tang, H.; Wang, L., et al., PLoS genetics 2013, 9, e1003923.
(15) Hylin, J. W., J Bacteriol. 1958, 76, 36-40.
(16) Yu, H.; Tang, H., et al., Sci. Rep. 2014, 4, 5397.
(17) Yu, H.; Tang, H., et al., J. Bacteriol. 2011, 193, 5541-2.
(18) Brandange, S.; Lindblom, L., et al., Acta Chem. Scand. B. 1983, 37, 617-622.
(19) Cooper, Z. D.; Narasimhan, D., et al., Mol. Pharmacol. 2006, 70, 1885-91.
(20) Hecht, S. S.; Hochalter, J. B., et al., Proc. Natl. Acad. Sci. U S. A. 2000, 97, 12493-7.
(21) Brandsch, R., Appl. Microbiol. Biotechnol. 2006, 69, 493-8.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

What is claimed is:

1. A method of treating nicotine addiction, reducing the risk of relapse of nicotine consumption, or treating nicotine poisoning in a subject in need thereof, comprising administering to the subject a therapeutically effective amount of a nicotine-degrading enzyme.

2. The method of claim 1, where the nicotine-degrading enzyme degrades nicotine into a compound selected from the group consisting of N-methylmyosmine and 4-(methylamino)-1-(pyridine-3-yl)butan-1-one.

3. The method of claim 1, wherein the nicotine-degrading enzyme is obtained from Pseudomonas putida.

4. The method of claim 1, wherein the nicotine-degrading enzyme is NicA2.

5. The method of claim 1, wherein the nicotine-degrading enzyme is a NicA2 variant that exhibits nicotine-degrading activity in vivo.

6. The method of claim 5, wherein the amino acid sequence of the NicA2 variant is at least 95% identical to SEQ ID NO:1.

7. The method of claim 5, wherein the amino acid sequence of the NicA2 variant is modified as compared to SEQ ID NO:1 to reduce immunogenicity in the subject.

8. The method of claim 5, wherein the amino acid sequence of the NicA2 variant is modified to enhance the catalytic efficiency or stability of the enzyme.

9. The method of claim 1, where the nicotine-degrading enzyme is conjugated or fused to a moiety that increases the circulating half-life of the enzyme in vivo.

10. The method of claim 9, wherein the moiety is selected from the group consisting of polyethylene glycol moieties, albumin moieties, and albumin-binding moieties.

11. The method of claim 9, wherein the moiety comprises an antibody Fc domain and/or a peptide moiety that mimics the properties of polyethylene glycol.

12. The method of claim 1, wherein the method comprises administering the nicotine-degrading enzyme by a route of administration selected from the group consisting of intranasally, orally, subcutaneously, intravenously, intraperitoneally, and intramuscularly.

13. The method of claim 1, wherein the method comprises administering an amount of nicotine-degrading enzyme of from 0.01 mg/kg to 100 mg/kg.

14. The method of claim 1, wherein the method comprises administering an amount of nicotine-degrading enzyme effective to achieve serum concentrations of nicotine-degrading enzyme of from about 0.1 μM to about 50 μM.

15. The method of claim 1, wherein the method comprises administering an amount of nicotine-degrading enzyme effective to achieve serum concentrations of nicotine-degrading enzyme of from about 0.5 μM to about 10 μM.

16. The method of claim 1, wherein the method is effective to reduce serum levels of nicotine in the subject.

17. The method of claim 1, wherein the method is effective to reduce brain levels of nicotine in the subject.

18. The method of claim 1, wherein the nicotine-degrading enzyme is administered once daily, once every two days, once every three days, twice weekly, once weekly, once every two weeks, once every three weeks, once every month, once every two months, once every three months, or once every six months.

19. The method of claim 1, wherein the method is effective to treat nicotine addiction, treat a nicotine-addiction related disorder, reduce the risk of relapse of nicotine consumption, promote smoking cessation, extend a duration of smoking abstinence in a subject who has quit smoking, increase a likelihood of long-term abstinence from smoking, and/or rescue a subject from relapse of nicotine consumption.

20. A method of degrading nicotine in vivo in a subject in need thereof, comprising administering to the subject a therapeutically effective amount of a nicotine-degrading enzyme.

21. A method of degrading nicotine in vivo in a subject in need thereof, comprising administering to the subject a therapeutically effective amount of an expression vector capable of expressing a nicotine-degrading enzyme in vivo.

22. A pharmaceutical composition comprising a therapeutically effective amount of a nicotine-degrading enzyme in a pharmaceutically acceptable carrier.

23. The composition of claim 22, where the nicotine-degrading enzyme degrades nicotine into a compound selected from the group consisting of N-methylmyosmine and 4-(methylamino)-1-(pyridine-3-yl)butan-1-one.

24. The composition of claim 22, wherein the nicotine-degrading enzyme is obtained from Pseudomonas putida.

25. The composition of claim 22, wherein the nicotine-degrading enzyme is NicA2 or a NicA2 variant that exhibits nicotine-degrading activity in vivo.

26. The composition of claim 25, wherein the nicotine-degrading enzyme is a NicA2 variant having an amino acid sequence at least 95% identical to SEQ ID NO:1.

27. The composition of claim 25, wherein the wherein the nicotine-degrading enzyme is a NicA2 variant having an amino acid sequence modified as compared to SEQ ID NO:1 to reduce immunogenicity.

28. The composition of claim 25, wherein the amino acid sequence of the NicA2 variant is modified to enhance the catalytic efficiency or stability of the nicotine-degrading enzyme.

29. The composition of claim 22, where the nicotine-degrading enzyme is conjugated or fused to a moiety that increases the circulating half-life of the nicotine-degrading enzyme in vivo.

30. The composition of claim 29, wherein the moiety is selected from the group consisting of polyethylene glycol moieties, albumin moieties, and albumin-binding moieties.

31. The composition of claim 90, wherein the moiety comprises an antibody Fc domain and/or a peptide moiety that mimicks the properties of polyethylene glycol.

32. The composition of claim 20, wherein the composition is formulated for administration by a route selected from the group consisting of intranasally, orally, subcutaneously, intravenously, intraperitoneally, and intramuscularly.

33. A pharmaceutical composition according to any one of claims 22-32, for use in treating nicotine addiction, reducing the risk of relapse of nicotine consumption, or treating nicotine poisoning in a subject in need thereof.

34. The composition for use according to claim 33, wherein the subject is in need of treatment for nicotine addiction, treatment of a nicotine-addiction related disorder, reduction of the risk of relapse of nicotine consumption, promotion of smoking cessation, extending a duration of smoking abstinence in a subject who has quit smoking, increasing a likelihood of long-term abstinence from smoking, and/or rescue from relapse of nicotine consumption.

35. The composition for use according to claim 33, wherein the composition is administered to the subject to provide an amount of nicotine-degrading enzyme of from 0.01 mg/kg to 100 mg/kg.

36. The composition for use according to claim 33, wherein the composition is administered to the subject in an amount effective to achieve serum concentrations of nicotine-degrading enzyme of at least 20 nM.

37. The composition for use according to claim 33, wherein the composition is administered to the subject in an amount effective to achieve serum concentrations of nicotine-degrading enzyme of from about 0.1 μM to about 50 μM.

38. The composition for use according to claim 33, wherein the composition is administered to the subject in an amount effective to reduce serum levels of nicotine in the subject.

39. The composition for use according to claim 33, wherein the composition is administered to the subject in an amount effective to reduce brain levels of nicotine in the subject.

40. The composition for use according to claim 33, wherein the composition is administered to the subject once daily, once every two days, once every three days, twice weekly, once weekly, once every two weeks, once every three weeks, once every month, once every two months, once every three months, or once every six months.

41. Use of a pharmaceutical composition according to any one of claims 22-32, in the preparation of a medicament for treating nicotine addiction, reducing the risk of relapse of nicotine consumption, or treating nicotine poisoning in a subject in need thereof.

42. The use according to claim 41, wherein the subject is in need of treatment for nicotine addiction, treatment of a nicotine-addiction related disorder, reduction of the risk of relapse of nicotine consumption, promotion of smoking cessation, extending a duration of smoking abstinence in a subject who has quit smoking, increasing a likelihood of long-term abstinence from smoking, and/or rescue from relapse of nicotine consumption.

43. The use according to claim 41, wherein the composition is administered to the subject to provide an amount of enzyme of from 0.01 mg/kg to 100 mg/kg.

44. The use according to claim 41, wherein the composition is administered to the subject in an amount effective to achieve serum concentrations of nicotine-degrading enzyme of from about 0.1 μM to about 50 μM.

45. The use according to claim 41, wherein the composition is administered to the subject in an amount effective to achieve serum concentrations of nicotine-degrading enzyme of from about 0.5 μM to about 10 μM.

46. The use according to claim 41, wherein the composition is administered to the subject in an amount effective to reduce serum levels of nicotine in the subject.

47. The use according to claim 41, wherein the composition is administered to the subject in an amount effective to reduce brain levels of nicotine in the subject.

48. The use according to claim 41, wherein the composition is administered to the subject once daily, once every two days, once every three days, twice weekly, once weekly, once every two weeks, once every three weeks, once every month, once every two months, once every three months, or once every six months.
